(19) United States
(12) Patent Application Publication
(10) Pub. No.: US 2011/0294135 A1
(43) Pub. Date: Dec. 1, 2011

(54) COMPOSITIONS AND METHODS FOR ANALYTE DETECTION AND QUANTITATION

(75) Inventor: Robert Henning Carlson, Seattle, WA (US)

(73) Assignee: BioDesic, LLC, Seattle, WA (US)

(21) Appl. No.: 12/671,897

(22) PCT Filed: Aug. 4, 2008
(86) PCT No.: PCT/US08/72110

§ 371 Date: Aug. 15, 2011

Related U.S. Application Data
(60) Provisional application No. 60/963,125, filed on Aug. 2, 2007.

(51) Int. Cl. C12Q 1/68; C07K 19/00; G01N 33/566

(52) U.S. Cl. 435/6.15; 436/501; 530/350; 530/387.3; 530/358

(57) ABSTRACT

The present invention relates to methods and compositions useful for the detection and quantitation of analytes. The present invention has particular applicability to the detection and quantitation of analytes in samples of biological origin or that interact with biological systems.

### CROSS REFERENCE TO RELATED APPLICATIONS

This application claims the benefit of U.S. Provisional Application No. 60/963,125, filed Aug. 2, 2007, the entire disclosure of which is hereby incorporated by reference in its entirety for all purposes.

### BACKGROUND OF THE INVENTION

1. Field of the Invention

The invention relates to methods and compositions useful for detection and quantitation of analytes, e.g., proteins and other biological materials. The present invention has particular applicability to the detection and quantitation of analytes in samples of biological origin or that interact with biological systems.

2. Description of the Related Art

Methods for detection of analytes, e.g., proteins, are routinely used in biotechnology and medicine. For example, immunoassays are commonly used for protein detection. An Enzyme Linked Immuno-Specific Assay (ELISA) is an immunoassay well-known in the art that can be used for protein detection. While ELISAs are in common use, they do not provide strictly quantitative results, and can be difficult to reproduce exactly. Moreover, the requirement of surface attachment of one or more components of the assay can reduce specificity and increase background. These detection and measurement challenges are general to problems requiring the identification of analytes, especially at small concentrations, in complex solutions or samples.

One alternative approach for detection of analytes is the use of so-called “tadpole” molecules as describe in Carlson and Burbulis (2002) (Robert Carlson and Ian Burbulis, “Chimeric Fusion Molecule for Sensitive Analyte Detection and Quantitation”, 2002, US Patent Application Publication No. 20030198973) and Burbulis et al (2005) (Burbulis et al, 2005 “Using protein-DNA chimeras to detect and count small numbers of molecules”, Nature Methods, 2(1): p. 31-37). Tadpoles are a chimeric fusion molecule consisting of a protein-based recognition domain covalently linked to a double stranded DNA (dsDNA) “barcode.” The recognition domain binds to the analyte in question; the DNA barcode is used to detect and quantify the tadpole. The tadpoles were used to quantify the presence of analytes in samples such as human blood. The method of detection in Burbulis et al (2005), in this case, was amplification and quantification of the dsDNA barcode component of tadpoles via real-time PCR. While sensitive, biochemical amplification techniques are subject to errors from noise and to the presence of contaminants. In the example of Burbulis 2005 great care was required to ensure no contaminants were present to produce spurious signal and, at low analyte concentrations, to ensure that the result of the amplification could be trusted to represent the number of analyte molecules present in the sample. In addition, the method of making the tadpoles involved a complicated approach using inteins to produce a protein moiety covalently attached to the DNA moiety.

The present invention solves these and other problems associated with sensitive and accurate analyte detection and quantitation in a sample. Further, the present invention is an improvement on the use of detector molecules, including tadpoles, by providing for a novel design and use.

### SUMMARY OF THE INVENTION

Disclosed herein is a composition for detection of a target analyte. The composition includes a binding domain, wherein the binding domain is capable of binding to the target analyte; and a linking domain, wherein the linking domain is capable of noncovalently binding to a detection moiety.

In one embodiment of the composition, the linking domain includes a nucleic acid binding protein. In one aspect the nucleic acid binding protein includes a zinc finger protein.

In another aspect, the linking domain includes more than one nucleic acid binding protein. In a related aspect the more than one nucleic acid binding protein includes 268 and NRE.

In another embodiment, the target analyte is capable of specifically binding a second target analyte. In one aspect, the target analyte is an IgG antibody. In another aspect, the second target analyte is an antigen.

In another embodiment, the binding domain includes LG. In another related embodiment, the binding domain can include LG, an antibody, a peptide, an antigen, a single chain fragment variable (scFv) protein, a viral coat protein, an H5N1 influenza viral coat protein, gp120, gp41, a DNA-binding protein, a protein that binds a target analyte of interest, or streptavidin.

In another embodiment, the target analyte includes an antibody, an IgG antibody, an Fc fragment of an antibody, a protein, a lipid, a polysaccharide, a metabolite, an inorganic component, an organic component, or an organic solvent.

In another embodiment, the composition further includes a detection moiety bound to the linking domain by a noncovalent bond. In one aspect, the detection moiety includes a polymer. In a related aspect, the polymer includes a nucleic acid oligomer. In another related aspect, the nucleic acid oligomer includes DNA. In another aspect, the DNA includes a nucleic acid restriction enzyme recognition site. In another aspect the detection moiety includes a nucleic acid binding site. In a related aspect, the nucleic acid binding site can be, e.g., a site where a primer or probe binds to the detection moiety. In another aspect, the detection moiety includes a nucleic acid restriction enzyme recognition site. In a related embodiment, the recognition site includes a nucleic acid restriction site or a nucleic acid polymerase binding site. In another aspect, the detection moiety includes a DNA binding site for a DNA binding protein. In another aspect the detection moiety includes a nucleic acid including the DNA binding site for 268-NRE.

In another embodiment, the detection moiety further includes a marker. In one aspect, the marker includes a gold particle, a metal particle, a silver particle, a dye, an isotope, a fluorophore, a magnetic particle, or a quantum dot. In another aspect, the detection moiety further includes more than one distinct marker.

Also described herein is a method for determining the presence or absence of an analyte in a sample, including: binding a composition for detection of a target analyte, including: a binding domain, wherein the binding domain is capable of binding to the target analyte; and a linking domain, wherein the linking domain is capable of noncovalently binding to a detection moiety, to a detection moiety to form a detector; acquiring the sample; combining the sample with the detector, wherein the combining results in binding of the analyte by the detector; and determining the presence of the analyte in the sample.

In one embodiment, the method further includes the step of separating the bound analyte from the sample. In another embodiment, the separation is performed by gel electrophoresis, capillary electrophoresis with a sieve medium, capillary electrophoresis without a sieve medium, elution, chromatography, hydrodynamic array, chemical composition-based separation, or charge-based separation. In another embodiment, the separation is based on the length of the detection moiety.

In another embodiment, the method further includes the steps of: analyzing the sample with a reader to determine the presence or absence of the analyte in the sample; and outputting data, wherein the data include the presence or absence of the analyte in the sample. In one aspect, the reader includes a cytometer, a gene chip, a camera, a gel, CE, or a microfluidic device.

In another embodiment, the method further includes the step of removing the detection moiety from the bound analyte. In one aspect, the removing is performed with a restriction enzyme, metal chelation, or heat.

In another embodiment the method further includes the steps of quantitating the analyte in the sample.

In another embodiment of the method, the sample is combined with more than one detector, the more than one detector including more than one distinct binding domain and more than one distinct detection moiety. In one aspect, each distinct detection moiety includes a distinct marker. In another aspect each distinct detection moiety includes a distinct length.

In another embodiment of the method, the determining is based on a mobility shift of the bound analyte upon separation.

In another embodiment of the method, the determining is performed with a polymerase. In one aspect, the determining is performed with polymerase chain reaction. In another aspect, the determining is performed with T7 polymerase.

In another embodiment, the method is performed in a liquid phase.

In another embodiment, the sample includes a biological sample.

In another embodiment, the separation step and the determining step are performed simultaneously.

In another embodiment, the method further includes binding a binder domain to the binding domain of the composition described above, wherein the binder domain is capable of specifically binding the analyte. In one aspect, the binder domain is an antibody. In another aspect, the analyte is an antigen.

Also described herein is a kit for detection of an analyte, including a composition for detection of a target analyte, including: a binding domain, wherein the binding domain is capable of binding to the target analyte; and a linking domain, wherein the linking domain is capable of noncovalently binding to a detection moiety, and instructions for use.

In one embodiment, the kit further includes a detection moiety. The detection moiety can be bound to the linking domain by a noncovalent bond.

In another embodiment, the kit further includes reagents for use of the composition.

In another embodiment, the kit further includes containers, wherein each container includes a distinct detection moiety. In one aspect each distinct detection moiety includes a distinct length. In another aspect, each distinct detection moiety includes a distinct sequence.

Also described herein is a method for determining the presence or absence of an analyte in a sample, including: acquiring the sample; combining the sample with the composition of the present invention (see above), wherein the combining results in binding of the analyte by the composition; and determining the presence of the analyte in the sample.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a schematic representing the components of a composition for detection of an analyte, e.g., a detector of the present invention. (1) Indicates the binding domain of the detector, which is attached to the linking domain (2). The detection moiety (3) is linked to the linking domain via a noncovalent bond (4). The resulting assembled detector is represented in the rest of the figures as item (5).

FIG. 2 illustrates the components of a “bound complex” or “bound detector”. The analyte (6) possesses at least one distinct region (e.g., 7 or 8) that can be bound by a detector (5) specific for the at least one distinct region. The noncovalent association of the analyte and the detector results in a bound complex (9).

FIG. 3 is a schematic demonstrating the detection of analyte in a sample by a mobility shift induced in detectors, where the difference in mobility is observed in a polyacrylamide or agarose gel. The shifted and unshifted detectors are both visualized via fluorescent dyes carried by the double stranded DNA portion of the complex. Lanes 1 and 4 are size standards; Lane 2 is the unbound detector molecule; and Lane 3 is the detector molecule bound to the analyte.

FIG. 4 is an image of a gel stained with SYBR Gold, which fluoresces when bound to DNA, demonstrating the principle of mobility shift for analyte detection. Lane 1 is a size standard, Hyperladder II. Lane 2 contains a 50-mer DNA detection moiety plus LG-268-NRE. Lane 3 contains a 50-mer DNA detection moiety plus LG-268-NRE plus Bovine IgG antibodies. Lane 4 contains an 80-mer DNA detection moiety. Lane 5 contains an 80-mer DNA detection moiety plus LG-268-NRE, where both the detection moiety and the detector complex are visible. Lane 6 contains an 80-mer DNA detection moiety plus LG-268-NRE plus Bovine IgG antibodies, where the unshifted detection moiety, the unbound detector complex, and the bound detector complex are visible as bands of varying width from the bottom to the top of the lane. Lane 7 contains a 100-mer DNA detection moiety. Lane 8 contains a 100-mer DNA detection moiety plus LG-268-NRE, where both the detection moiety and the detector complex are visible. Lane 9 contains a 100-mer DNA detection moiety plus LG-268-NRE plus Bovine IgG antibodies, where the unshifted detection moiety, the unbound detector complex, and the bound detector complex are visible as bands of varying width from the bottom to the top of the lane.

FIG. 5 is a schematic demonstrating the same assay as FIG. 2, except that there are two distinct detectors bound to distinct regions of the analyte, further altering the mobility of the complex and simultaneously increasing the signal from fluorophores attached to the DNA detection moiety. Lanes 1 and 4 are size standards; Lane 2 is the unbound detector molecules; and Lane 3 is the detector molecules bound to the analyte.

FIG. 6 is a schematic demonstrating a multiplexed, quantitative assay for components of a complex solution. The intensity of the band in the gel is a direct physical measurement of the number of bound complexes in that band, and thus of the number of analytes bound by detectors with detection moieties of a certain length. Lanes 1 and 5 are size standards; Lane 2 is the unbound detector molecule; Lane 3 is all detector molecule bound to their respective analytes; and Lane 4 is analysis of a complex solution comprising differing numbers of analytes, where the intensity of the band determines the number of bound complexes in the band and thus the number of analytes present in the sample.

FIG. 7 is a schematic of a detection moiety, in this case dsDNA. The detection moiety can contain functional regions, depicted as A, B, and C, which might, for example, respectively be the binding sequence for NRE-268, a recognition site for the restriction enzyme EcoR1, and a promoter site for T7 DNA-dependent RNA polymerase.

FIG. 8 illustrates a detector complex, for example comprised of a dsDNA detection moiety and LG-268-NRE. The addition of a restriction enzyme to a solution containing the detector complex will result in cutting of the dsDNA detection moiety at the location of any present restriction sites, as represented by the dashed line (A). This will leave free in solution the cleaved detector (B) and dsDNA fragments (C), of lengths corresponding to the distance between the restriction site and the end of the original dsDNA strand. These free dsDNA fragments can be separated and identified using techniques well known in the art.

FIG. 9 is an image of a gel stained with SYBR Gold. Lanes 1-3 are size standards, Bioline Hyperladder I, Bioline Hyperladder II, and Bioline Hyperladder III, respectively. Lane 4 contains a 100-mer DNA detection moiety plus LG-268-NRE. Lane 5 contains a 100-mer DNA detection moiety plus LG-268-NRE. Lane 6 contains a 100-mer DNA detection moiety plus LG-268-NRE plus Bovine IgG antibodies. Lane 7 contains a 100-mer DNA detection moiety plus LG-268-NRE plus bovine IgG antibodies. Lane 8 contains a 100-mer DNA detection moiety plus LG-268-NRE plus anti-streptavidin A (StrA) antibodies. Lane 9 contains a 100-mer DNA detection moiety plus LG-268-NRE plus anti-streptavidin A (StrA) antibodies plus Streptavidin A. Lane 10 contains a 100-mer DNA detection moiety plus LG-268-NRE plus anti-PSA IgG control antibodies (Biomeda) that shows no binding.

### DETAILED DESCRIPTION OF THE INVENTION

The present invention provides compositions, e.g., detector molecules, for detection of analytes, e.g., proteins, and methods of using the compositions. The detector molecule includes a binding domain and linking domain. The binding domain, typically a protein moiety, e.g., an antibody, an scFv fragment, or antibody fragment, is capable of binding the analyte. The linking domain, typically a protein moiety, e.g., a DNA-binding protein, is capable of binding to a detection moiety. The detection moiety is typically a nucleic acid, e.g., a DNA molecule that includes the sequence recognized by the linking domain. Once the detector molecule has bound an analyte in a sample, the detector molecule plus analyte can be detected via detection of the detection moiety to which the detector molecule is bound via, e.g., a gel-shift assay. In another aspect of the present invention, the detection moiety can be separated from the bound detector molecule and quantified separately.

One advantage of the present invention is transduction from one signal, a target analyte, to another signal that is easier to detect and quantify, such as DNA. A detector molecule bound to a target analyte thus serves to transduce the presence of the analyte into the presence of the detection moiety.

Another advantage of the present invention is that the interaction between the detection moiety and the linking domain of the detector molecule is noncovalent. The noncovalent chemistry allows for simpler production and construction of detector molecules, and also enables construction of bound complexes serially; i.e., the adding of detector moieties after the binding domain is already bound to the analyte. This is in contrast to “tadpole” molecules, where the interaction between the linking domain and the detection moiety is covalent.

In another advantage, the present invention enables detection via gel-shift assays in addition to detection via nucleic acid amplification. The shift in mobility imparted by binding of an analyte to the detector molecule is employed to detect analytes. Comparing the intensity of unshifted and shifted bands, i.e., unshifted and shifted detector molecules, can provide a method to quantify the analyte in the sample. Therefore, using a detection method that enables counting the number of DNA molecules provides the number of corresponding target analyte numbers.

In another advantage, detector molecules are used to label pairs of antibodies that bind a target, where the coincidence of those labels within a temporal or spatial window indicates the presence of the target analyte. In another advantage, the detection moieties are separated from the detector complex, for example by elution or through the use of restriction enzymes, thereby leaving detection moieties of different lengths or sequences in direct proportion to the number of each analyte species in solution. These detection moieties can then be separated and quantified according to well-known techniques.

### DEFINITIONS

Terms used in the claims and specification are defined as set forth below unless otherwise specified.

“Detector,” “detector molecule,” and “composition for detection” are used interchangeably, and refer to the herein described composition for detection of a target analyte. The terms are used to refer to a composition both with and without the detector moiety, e.g., the nucleic acid to which the linking domain binds. “Detection” of an analyte includes determining the presence or absence of an analyte. Depending on the application, detection can quantify the analyte, e.g., determine the amount of an analyte in a sample.

“Target analyte” as described in further detail below, is material to be detected, e.g., an IgG antibody, in a sample.

“Binding domain” as described in further detail below, refers to a moiety of the detector composition that specifically recognizes and binds to the target analyte. One example of a binding moiety is LG, a protein that recognizes and binds to IgG.

“Linking domain” refers to a moiety of the detector composition that can non-covalently bind to the detection moiety. One example of a linking domain is a DNA binding protein, e.g., 268-NRE. Further details and examples of linking domains are described below.

“Detection moiety” is typically a nucleic acid polymer, e.g., DNA that is recognized and bound by the linking domain. A nucleic acid with the sequences for the 268-NRE binding site is an example.

“LG” refers to an immunoglobulin binding protein that can be used as a binding domain in the present invention. The Genbank accession number of LG is S50809.

“268” refers to a zinc finger protein that can be used as a linking domain in the present invention. The 268 Zinc Finger is accession number (for the peptide sequence): 1AAY_A. References for 268 include: Pavletich, N. P. and Pabo, C. O. “Zinc finger-DNA recognition: crystal structure of a Zif268-DNA complex at 2.1 A”, Science 252 (5007), 809-817 (1991).

“NRE” refers to a three-finger peptide that specifically recognizes a nuclear hormone response element and can be used as a linking domain in the present invention. NRE is described in more detail in Kim, J. and Pabo, C., “Getting a handhold on DNA: Design of poly-zinc finger proteins with femtomolar dissociation constants”, Proc. Natl. Acad. Sci. USA, Vol. 95, pp. 2812-2817, March 1998.

“Nucleic acid” refers to a molecule that can form a chain of monomeric nucleotides. Nucleic acids can include DNA and RNA.

“Protein” and “polypeptide” are used interchangeably and refer to an organic molecule made of amino acids arranged in a chain and joined together by peptide bonds between the carboxyl and amino groups of adjacent amino acid residues.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

The term “mammal” as used herein includes both humans and non-humans and include but is not limited to humans, non-human primates, canines, felines, murines, bovines, equines, and porcines.

It must be noted that, as used in the specification and the appended claims, the singular forms “a,” “an” and “the” include plural referents unless the context clearly dictates otherwise.

All gene descriptions and accession numbers refer to sequences available in GenBank on Aug. 4, 2008. It is understood the invention is not limited to the exemplary sequences presented and/or referred to herein

Compositions of the Invention

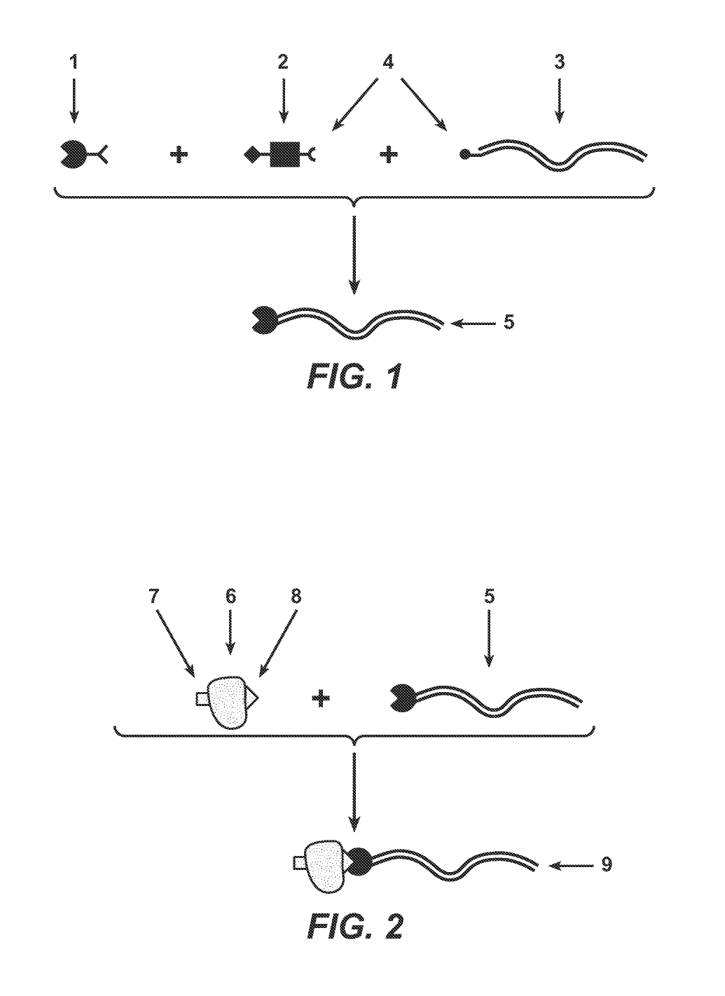

The compositions of the present invention, e.g., the detector molecules, are typically comprised of two or more domains. At least one binding domain serves to recognize and noncovalently bind target analytes such as peptides or other molecules, while at least one linking domain serves to noncovalently recognize and bind a detection moiety such as DNA (FIG. 1). The DNA contains a sequence recognized by the linking domain. The compositions can be used to link target analytes, e.g., proteins and small molecules to nucleic acids, e.g., DNA via programmable specific binding, where the identity of the binding and linking domains determine the components to be linked. An example of a composition of the invention is LG-268-NRE, composed of the binding domain protein LG, comprised of peptides L and G, and of the zinc-finger DNA-binding proteins Zif-268 and NRE, as the linking domain. LG-268-NRE can be used to link IgG antibodies to DNA containing the binding sites for Zif-268 and NRE.

Other examples of binding domains include streptavidin, single-chain fragment antibodies, or any peptide that recognizes targets, e.g., analytes of interest. Further descriptions of binding domains are described herein.

Linking domains can be any nucleic acid binding protein, such as, e.g., lambda repressor or mammalian transcription factors. Further descriptions of linking domains are found below.

Detection moieties are typically nucleic acids, e.g., DNA. The detection moieties can be double-stranded DNA of a defined length or sequence, which can contain at least one binding site for the linking domain, e.g., the DNA-binding protein. DNA fragments can contain additional functional sequences such as recognition sites for restriction enzymes or DNA-dependent polymerases. Composite structures comprised of a target analyte, a composition of the present invention, and DNA can be used as detectors in e.g., mobility shift assays, and constitute examples of designed, nanofabricated devices. In another aspect, detector molecules comprising dsDNA and fusion proteins such as LG-268-NRE, can be detected via amplification with e.g., PCR or T7 polymerase.

Because a composition can comprise linking domains that bind to different DNA sequences, the assembly of various compositions can be programmed by mixing and matching different binding domains with different linking domains. Thus, one composition can be comprised of LG and 268-NRE, while a second can be comprised of LG and a Tet repressor protein. In another aspect, the second composition can be comprised of LG and a different zinc finger protein. Similarly, one composition can be comprised of an influenza viral coat protein and 268-NRE and a second composition can be comprised of LG and a Tet repression protein. In use, these compositions can be used to enable multiplexed analysis using multiple distinct detector molecules comprising multiple distinct detection moieties, one example of which is, highly specific detection of antibodies against a viral coat protein.

Binding Domain

The detector molecules of the invention include a binding domain. A binding domain typically binds to an analyte, e.g., a protein of interest. In one embodiment, a binding domain can encompass monoclonal antibodies, polyclonal antibodies, antibody fragments (e.g., Fab, Fab′, F(ab′)₂, Fv, Fc, etc.), chimeric antibodies, single chain Fvs (ScFvs), mutants thereof, fusion proteins comprising an antibody portion, a peptide, an antigen, LG, streptavidin, and any other polypeptide that comprises an antigen recognition site of the required specificity (including antibody mimetics). See, e.g., Xu et al, Chem. Biol. 2002 Aug. 9(8):933-42. Other preferred binding domains can include: Streptavidin (Genbank accession number: X03591), including engineered monomeric versions derived from the naturally occurring protein; Protective Antigen (GeneID: 2820165), for use, e.g., in antibody fishing as a diagnostic for exposure to Bacillus anthracis (anthrax); Hemaglutinnin (HA) (GeneID: 3654620), for use, e.g., in antibody fishing as a diagnostic for exposure to influenza A H5N1 subtypes; Neuraminidase (NA) (GeneID: 3654619), for use, e.g., in antibody fishing as a diagnostic for exposure to influenza A H5N1 subtypes; 14B7 scFv, and anti-protective antigen recombinant antibody fragment (J. Maynard, et al, Nature Biotechnology 20, 597-601 (2002)), for use, e.g., in diagnosis of anthrax infections or exposure.

The antibodies can be murine, rat, rabbit, chicken, human, or of any other origin, including humanized antibodies. Binding domains can be made recombinantly and expressed using any method now known or later discovered in the art. Binding domains can be made recombinantly by phage display technology. For examples of these expression and production methods see e.g., U.S. Pat. Nos. 5,565,332; 5,580,717; 5,733,743 and 6,265,150; and Winter et al., Annu. Rev. Immunol. 12:433-455 (1994).

In another embodiment, binding domains can be expressed as a fusion protein with a linking domain. Linking domains are discussed in greater detail below.

As used herein, the term “antibody” encompasses not only intact polyclonal or monoclonal antibodies, but also fragments thereof (such as Fab, Fab′, F(ab′)₂, Fv), single chain (scFv), mutants thereof, fusion proteins comprising an antibody portion, and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

“Fv” is an antibody fragment that contains a complete antigen-recognition and -binding site. In a two-chain Fv species, this region consists of a dimer of one heavy and one light chain variable domain in tight, noncovalent association. In a single-chain Fv species, one heavy and one light chain variable domain can be covalently linked by a flexible polypeptide linker such that the light and heavy chains can associate in a dimeric structure analogous to that in a two-chain Fv species. It is in this configuration that the three complementarity determining regions (CDRs) of each variable domain interact to define an antigen-binding specificity on the surface of the VH-VL dimer. However, even a single variable domain (or half of a Fv comprising only 3 CDRs specific for an antigen) has the ability to recognize and bind antigen, although generally at a lower affinity than the entire binding site.

A “monoclonal antibody” refers to a homogeneous antibody population wherein the monoclonal antibody is comprised of amino acids (naturally occurring and non-naturally occurring) that are involved in the selective binding of an antigen. A population of monoclonal antibodies (as opposed to polyclonal antibodies) are highly specific, in the sense that they are directed against a single antigenic site. The term “monoclonal antibody” encompasses not only intact monoclonal antibodies and full-length monoclonal antibodies, but also fragments thereof (such as Fab, Fab′, F(ab′)₂, Fv), single chain (scFv), mutants thereof, fusion proteins comprising an antibody portion, and any other modified configuration of the immunoglobulin molecule that comprises an antigen recognition site of the required specificity and the ability to bind to an antigen (see definition of antibody). It is not intended to be limited as regards to the source of the antibody or the manner in which it is made (e.g., by hybridoma, phage selection, recombinant expression, transgenic animals, etc.).

In another embodiment, binding domains can be blocked using blocking agents such as, e.g., serum or serum diluted in phosphate buffered saline (PBS) and other blocking agents known in the art.

The choice of binding domains depends on the application and the analyte(s) to be detected in a sample. Selection and use of binding domains is discussed in greater detail below.

As described herein, a binding domain can bind a peptide of 2 or more consecutive (i.e., sequential) amino acids. It is understood that the amino acid(s) forming an analyte can be linear or branched, and can comprise an amino acid(s) that has been modified naturally or by intervention; for example, disulfide bond formation, glycosylation, lipidation, acetylation, phosphorylation, or any other manipulation or modification, such as conjugation with a labeling component. The amino acid(s) forming an analyte can further encompass, for example, one or more analogs of an amino acid (including, for example, unnatural amino acids, etc.), as well as other modifications known in the art.

In some embodiments, the binding domain binds its cognate binder with an affinity of binding reaction of at least about 10⁻⁷ M, at least 10⁻⁸ M, or at least about 10⁻⁹ M, or tighter. In some embodiments, a binding interaction will discriminate over adventitious binding interactions in the reaction by at least two-fold, at least five-fold, at least 10- to at least 100-fold or more.

It is understood that other analyte binding agents can be used, in addition to the binding domain described herein. As an example, LG can be used as a binding domain of the present invention. LG can be bound to the Fc portion of an IgG antibody. The IgG antibody can be specific for any known target protein. The bound LG/IgG antibody combination can then be used to detect the presence or absence of an analyte of interest in a sample.

Linking Domain

The detector molecules of the invention include a linking domain. The linking domain recognizes and noncovalently binds to a detection moiety, e.g., a nucleic acid molecule. The detection moiety is discussed in greater detail below. In one embodiment, the linking domain of the present invention is a nucleic acid binding protein and the detection moiety is a nucleic acid oligomer. Nucleic acid binding proteins can be naturally occurring or synthetic molecules. A number of naturally occurring or synthetic molecules recognize nucleic acids under physiological conditions, and these are generally well-known in the art. Examples of such molecules include transcription factors (e.g., such as zinc-finger proteins (ZFPs)), Helix-Turn-Helix motif proteins (e.g., GATA-1), immunoglobulin motif proteins (e.g., NFκB, NFAT), lambda repressor, and polyamides, such as oligomeric heterocyclic minor groove binders (MGBs). Advantages of these nucleic acid binding proteins include: 1) they bind to double-stranded nucleic acids, and 2) they do so under almost the same conditions as proteins used to bind to other proteins and other molecules.

Typically the linking domain is recombinantly expressed as a fusion protein with the binding domain. For example, the binding domain LG can be fused to the linking domain 268-NRE as a genetic sequence and expressed by a cell as a fusion protein.

In one embodiment, the linking domain is a ZFP. ZFPs are transcription factors typically found in eukaryotes (e.g., in yeasts, plants, and mammals), that bind to nucleic acids. A zinc finger can consist of two antiparallel β strands, and an α-helix. The zinc ion typically stabilizes this domain type. One very well explored subset of zinc-fingers (the C₂H₂ class) comprises a pair of cysteine residues in the beta strands and two histidine residues in the α helix which are responsible for binding a zinc ion. The two other known classes of zinc finger proteins are the C₄ and C₆ classes. Zinc fingers are important in regulation because when interacted with DNA and zinc ion, they provide a unique structural motif for DNA-binding proteins. Many transcription factors (such as, e.g., Zif268), regulatory proteins, and other proteins that interact with DNA contain zinc fingers. These proteins typically interact with the major groove along the double helix of DNA in which case the zinc fingers are arranged around the DNA strand in such a way that the α-helix of each finger contacts the DNA, forming an almost continuous stretch of α-helices around the DNA molecule. The binding specificity of zinc fingers for 3-4 base pairs of a nucleic acid can be conferred by a short stretch of amino acid residues in the α-helix. The primary position of the amino acid residues within the α-helix interacting with the DNA are typically at positions −1, 3 and 6 relative to the first amino acid residue of the α-helix. Other amino acid positions can also influence binding specificity by assisting amino acid residues to bind a specific base or by contacting a fourth base in the opposite strand, causing target-site overlap.

Appropriate pairs of ZFPs can be selected using: Morgan L. Maeder, et al, Rapid “Open-Source” Engineering of Customized Zinc-Finger Nucleases for Highly Efficient Gene Modification, Molecular Cell, Vol 31, 294-301, 25 Jul. 2008, herein incorporated by reference for all purposes. Thus, one of ordinary skill can construct a library of linking domains comprising ZFP pairs that enable construction of detector molecules that bind millions of different distinguishable DNA sequences. This can enable, for example, a DNA array to be used to anchor binding domains that could target large numbers of different analytes.

In another embodiment, the linking domain is 268-NRE. 268-NRE represents a preferred example of a ZFP to be used as a linking domain in the composition. Other preferred linking domains of the present invention include: Tetracycline repressor protein (GeneID: 5148505), or engineered high affinity versions of the same; Lac repressor (GeneID: 4714237), or engineered high affinity versions of the same; and Lambda repressor (cI) (X00166), or engineered high affinity versions of the same. In a related embodiment, the linking domain is an oligomer of DNA binding proteins, e.g., comprising one or more Tetracycline repressor proteins, one or more Lac repressor proteins, one or more Lambda repressor proteins, or one or more engineered high affinity versions of any of these proteins.

Detection Moiety

The detector molecules of the invention recognize and noncovalently bind to a detection moiety. The detection moiety can be any molecule generally appropriate for the detector molecule. In general, the detection moiety is a polymer, e.g., a nucleic acid molecule. For example, in one embodiment, the DNA-binding protein moiety of the detector molecule recognizes and binds to a DNA molecule that includes the DNA binding recognition site. Depending on the application and the method used for detection, the detection moiety can include a marker, e.g., a label.

In one embodiment, the detection moiety of the present invention can be a polymer. Polymers can include a linear nucleic acid oligomer, a plasmid nucleic acid, or a polypeptide. In a related embodiment, nucleic acids of the detection moiety can include DNA and RNA. In another related embodiment, the DNA can include a restriction site or a recognition site for a DNA-dependent polymerase. In addition, nucleic acids of the detection moiety can be recombinant or synthetic.

A recombinant nucleic acid is prepared by first isolating the nucleic acid of interest. The isolated recombinant nucleic acid can be ligated into a suitable cassette, vector, or plasmid. Methods for preparing a recombinant nucleic acid are known by those skilled in the art (see Sambrook et al., Molecular Cloning: A Laboratory Manual (2d ed. 1989)).

In addition to the recombinant nucleic acids discussed above, synthetic nucleic acids or oligonucleotides can also be used in the invention. As a general point regarding the nucleic acids used in the invention, those of skill in the art recognize that the nucleic acids used in the invention include both DNA and RNA molecules, as well as synthetic, non-naturally occurring analogs of the same, and heteropolymers, of deoxyribonucleotides, ribonucleotides, and/or analogues of either. The particular composition of a nucleic acid or nucleic acid analogue will depend upon the purpose for which the material will be used and the environment in which the material will be placed. Modified or synthetic, non-naturally occurring nucleotides have been designed to serve a variety of purposes and to remain stable in a variety of environments, such as those in which nucleases are present, as is well known in the art. Modified or synthetic non-naturally occurring nucleotides, as compared to naturally occurring ribo- or deoxyribonucleotides can differ with respect to the carbohydrate (sugar), phosphate bond, or base portions of the nucleotide, or can even contain a non-nucleotide base (or no base at all) in some cases (see, e.g., Arnold et al., PCT patent publication no. WO 89/02439). For example, the modified or non-naturally occurring nucleic acids of the invention can include biotinylated nucleic acids, O-methylated nucleic acids, methylphosphonate backbone nucleic acids, phosphorothioate backbone nucleic acids, or polyamide nucleic acids. Synthetic nucleic acids can also be ligated into a suitable cassette, vector, or plasmid, as is generally well-known in the art.

In another embodiment, the nucleic acids of the detection moiety can contain a nucleic acid binding site. Such sites can be used, e.g., for binding a primer or a probe. The nucleic acids of the detection moiety can include one or more recognition site. Recognition site includes, e.g., a nucleic acid restriction enzyme site and/or a nucleic acid polymerase binding site. Such recognition sites can be used for cleaving the nucleic acid with a restriction enzyme or amplifying the nucleic acid using, e.g., PCR or T7 polymerase. In another embodiment, the detection moiety can include a DNA binding site for 268-NRE or other known ZFPs.

In another embodiment, nucleic acids of the detection moiety can have various lengths. The nucleic acids can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 80, 50 to 100, 101 to 500, 501 to 1000, 1001 to 5000, 5001 to 10000, or more nucleic acid bases in length. The variable length of the nucleic acids can allow, e.g., detection, differentiation, quantitation, and/or multiplexing of the present invention, as is discussed in more detail below.

Markers

In one embodiment of the present invention, the detection moiety can carry a marker detectable by means such as e.g., color, fluorescence or electromagnetic signals. In one aspect of the present invention where the detection moiety is a nucleic acid comprising DNA molecule, the DNA molecule can be labeled with dyes whose fluorescence is dramatically increased when bound to the DNA. This enables the detection of small amounts of DNA, and even of individual DNA molecules, with respect to normal background noise. In another aspect, the marker can comprise metal nanoparticles detectable by their light scattering or electrical properties, quantum dots detectable by their optical, electrical, or magnetic properties, or magnetic domains detected via induced currents.

Markers suitable for use in the present invention are compounds that are generally capable of producing, either directly or indirectly, a detectable signal. Examples of the types of markers that can be used with the methods of the invention include, e.g., fluorescent or colored dyes, isotopic labels, enzymes, immune labels (e.g., antibodies or antigens), gold particles, fluorophores, magnetic particles, and quantum dots. The markers can be incorporated into a detection moiety. The marker can directly or indirectly provide a detectable signal. Any method known in the art for conjugating and/or binding a marker to a detection moiety can be used.

In one embodiment, fluorescent labels are used as markers. Markers in the present invention can include, but are not limited to, e.g., Texas red (commercially available from Molecular Probes), and Alexa647 (commercially available from Molecular Probes). Other fluorescent or chemiluminescent compounds that can be used are, e.g., fluorescein isothiocyanate, rhodamine, and luciferin. In another embodiment, the markers are radiolabels, e.g., ³H, ¹²⁵I, ³⁵S, ¹⁴C, or ³²P. One of skill in the art will appreciate that the marker can be an enzyme (e.g., horse radish peroxidase, alkaline phosphatase, beta-galactosidase and others commonly used in, e.g., an ELISA); biotin for staining with labeled streptavidin conjugate; magnetic beads, and labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

In another embodiment, the detection moiety can be labeled with a plurality of markers. The plurality can include 2, 3, 4, 5, 6, 7, 8, 9, 10 or more markers, as is deemed necessary by one of ordinary skill for using the present invention to detect an analyte in a sample of interest to one of ordinary skill in the art.

Analyte

Analytes and target analytes of the present invention can include any protein, antibody, IgG antibody, Fc portion of an antibody, lipid, polysaccharide, metabolite, inorganic component, organic component, or organic solvent of interest. In one embodiment, the analyte or target analyte can bind to a second analyte or second target analyte. The second analyte can be any known protein, antibody, IgG antibody, lipid, polysaccharide, metabolite, inorganic component, organic component, or organic solvent.

Kit

The present invention further comprises the use of a kit or kits to detect and/or quantitate an analyte in a sample of interest. The kits of the present invention can comprise, e.g., instructions, reagents, and other materials necessary for use of the composition, and/or detection moieties, and/or detectors of the present invention.

Methods of the Invention

Detection of Target Analytes Bound to the Composition

In one embodiment, the detector molecules of the invention are used in a method for detection of an analyte in a sample. In one aspect, a detector is formed by binding a detection moiety to a composition of the present invention. In a related aspect, the detector is combined with an acquired sample and allowed to bind an analyte in the sample. In another aspect, the bound detector is used to determine the presence or absence of the analyte in the sample.

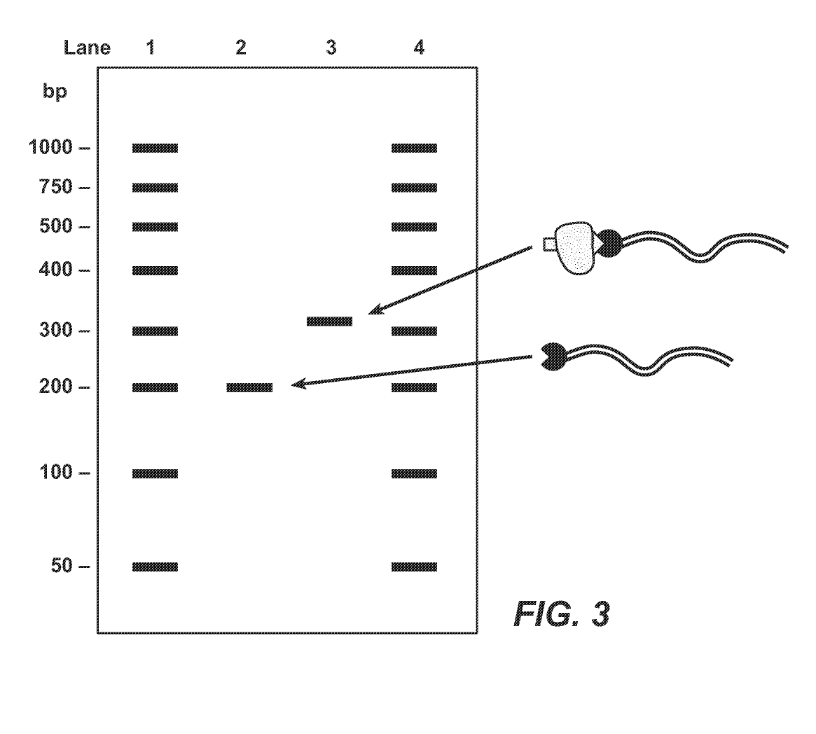

In another embodiment of the present invention, detection is performed through the binding of analytes to a binding domain while both components are freely suspended in a solution. In one aspect, the binding of the analyte to the binding domain of the composition changes the effective mass and/or size of the composition (FIG. 2), thus altering hydrodynamic or electrophoretic mobility (FIG. 3). In another aspect, the presence of a single analyte can be detected through its influence on a large number of markers on the composition. One of ordinary skill in the art would recognize this influence as a transduction of the presence of the analyte into the detection moiety. The number of mobility shifted compositions can therefore be representative of the number of analytes in a given sample. In another aspect, the method of detection can be used, for example, in complex solutions without requiring nucleic acid amplification. It is an additional aspect of the present invention to enable solution phase assays, wherein the detection step is accomplished without extensive purification, which can alleviate the need to immobilize the compositions, analytes, or other assay components on a solid surface.

Plurality of Compositions

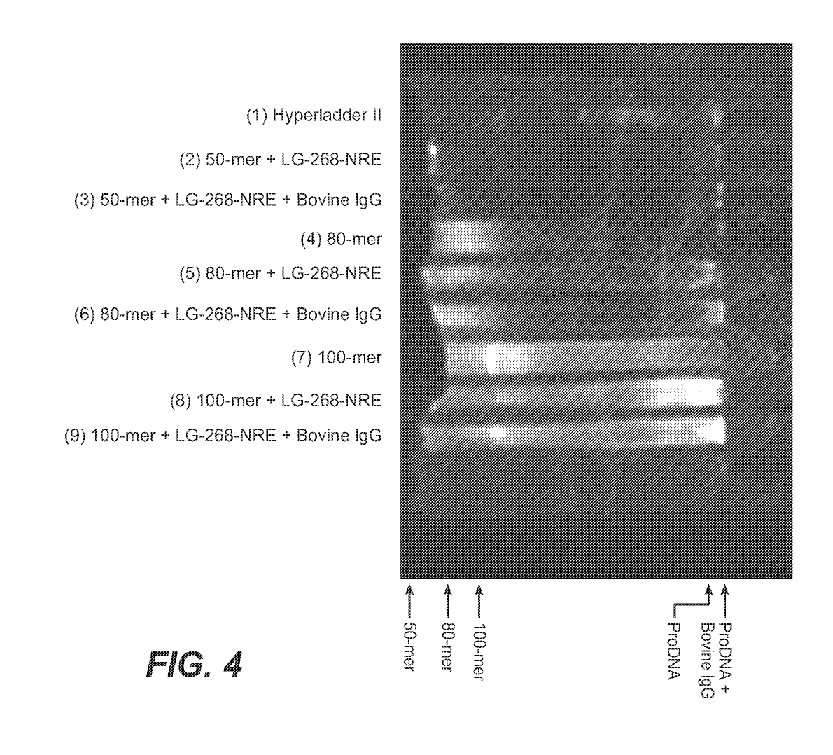

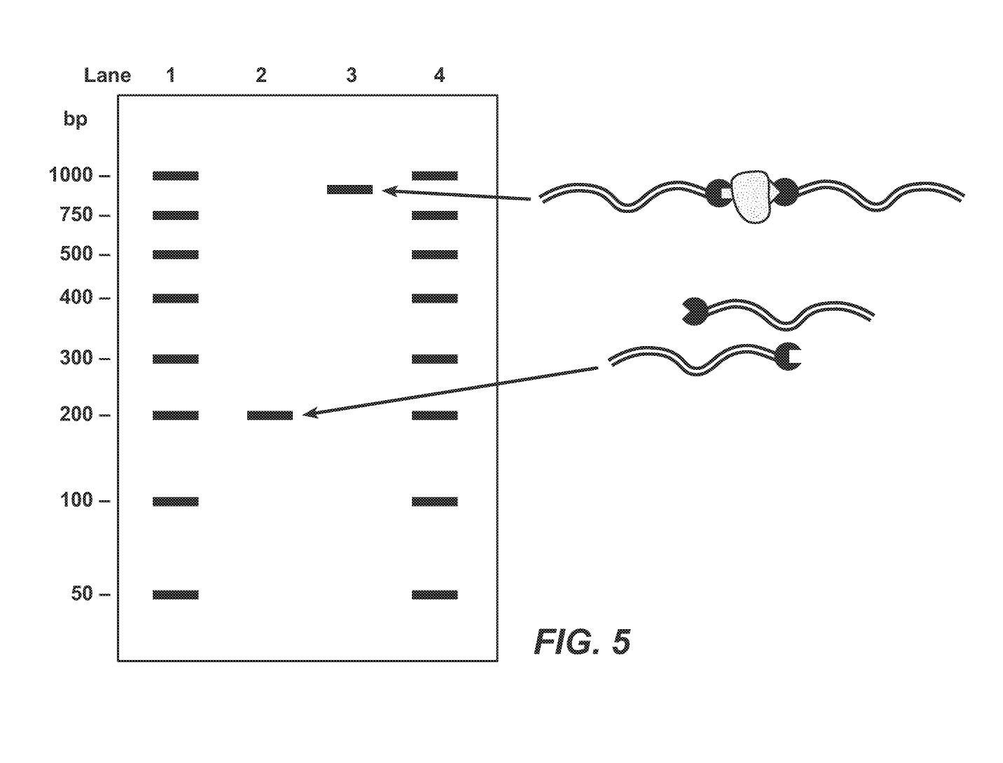

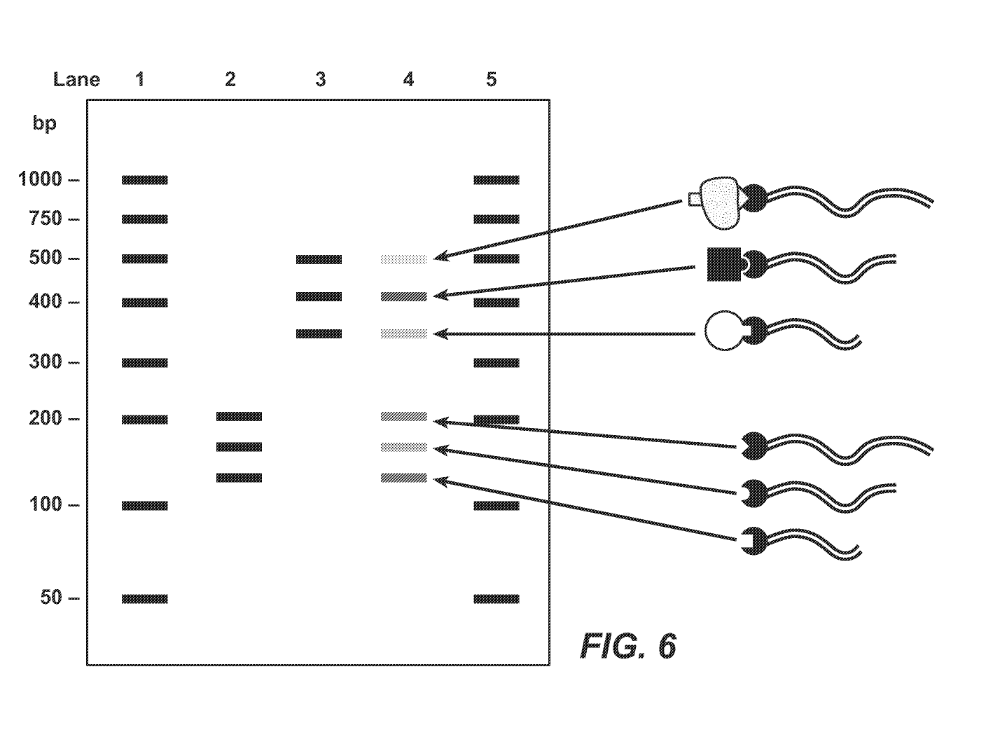

Methods of the present invention can be performed wherein more than one distinct composition binds a single analyte; i.e. the more than one distinct composition recognizes more than one unique stereochemical features of an analyte (FIG. 4). Methods can also be performed using a solution of two or more distinct species of compositions that each recognize different and distinct analytes, wherein each distinct species of composition can be characterized by a different mobility, and whose resulting mobility can be modified by the analytes in such a way that all species of mobility shifted and bound compositions are distinguishable from each other (FIG. 5). In one aspect, each distinct composition can comprise a distinct marker or markers to allow distinction between each distinct composition upon detection. These methods will allow multiplexing of the present invention (FIG. 6).

In one aspect, the analyte contains regions subject to binding by distinct compositions containing different binding domains, allowing the characteristics of the bound complex to be further modified. In another aspect, the number of detectable markers can be increased by a factor equal to the number of bound distinct compositions. For example, the presence of antibodies produced by the immune system can be detected using one composition comprising a binding domain constructed using the antigen the antibody recognizes, and a second composition comprising a binding domain that binds all antibodies. This would produce a bound complex comprising an analyte and two distinct compositions. The presence of both distinct detector moieties can then be measured directly, for example, by coincidence detection in hydrodynamic flow or simply via a change in electrophoretic mobility (FIG. 5).

Separation

In another embodiment of the present invention the bound analyte can be separated from the sample. In one aspect, bound compositions are first purified from those that remain unbound, for example, via mobility shift or using a second immobilized affinity reagent that captures bound compositions and allows removal of unbound versions. In one aspect, the detection moiety can be separated from compositions by elution or via the use of restriction enzymes, leaving detection moieties of various lengths or sequences in solution whose number is proportional to that of an analyte(s). The separated solution can then be analyzed via known techniques (described below) to determine the presence and number of each detection moiety length, thereby serving as a measure of analyte quantity.

In one aspect, the detection moiety possesses physical features that render it differentiable via some chromatographic technique, such as electrophoresis or high-pressure liquid chromatography (HPLC). The bound compositions can be fractionated or separated from the rest of the sample by e.g., electrophoresis in a gel, capillary electrophoresis with or without a sieving medium, hydrodynamically within a chromatographic column or microfabricated array, or by total chemical composition, charge, etc. In another aspect, the detection moiety is detected while still a part of the bound complex, and the mobility of the composition can be resolved using e.g., the above described techniques. In another aspect, mobility is modified by the presence of an analyte, and will result in a detectable difference between the mobility of bound and unbound complexes (FIG. 3). In another aspect, the detection moiety is separated from bound complex before detection, and then sensitive and high resolution instruments can be used to quantify each moiety in solution. This method can be used to analyze a plurality of analytes in the same sample because the mobility of detection moieties comprised of DNA differing in length by as little as one base pair can be resolved e.g., in gels or via capillary electrophoresis (CE). In one aspect of the present invention, the steps of separation and/or purification are combined with detection.

The entire bound detector need not be chromatographically separated simultaneously. A detection moiety can be separated from a bound detector, either by elution or, in the case that the detection moiety is DNA and contains a recognition site for a restriction enzyme, by cutting the detection moiety with the enzyme and separating the resulting fragments chromatographically, as described above.

Detection

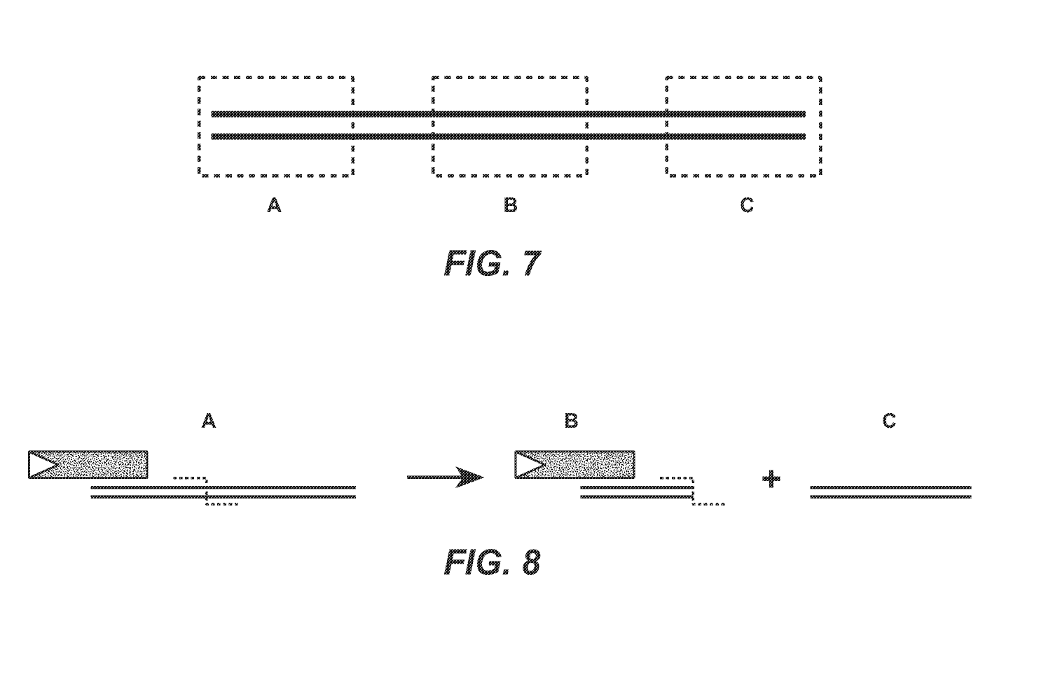

In one embodiment, different DNA detection moieties can include, for example, different lengths, different sequences, and/or markers that are physically detectable through characteristics such as fluorescence spectrum or paramagnetism (FIG. 7). The detection moieties can be detected using a variety of techniques known in the art including: polymerase, PCR, T7 polymerase, restriction enzyme digestion, and mobility assays. In one aspect, the present invention comprises a method of transducing the presence of the analyte into 1) an easily observably mobility shift of the composition and 2) decorating the composition with sufficient markers (e.g., gold particles, fluorophores, quantum dots, etc.), such that the bound composition can be observed and quantified by some means, e.g. PCR, enzymatic digestion, and/or separation. In another aspect, the present invention includes removing detection moieties from bound compositions via one or more restriction enzymes (FIG. 8) and detecting and quantifying only the resulting detection moieties rather than the entire bound composition. In another aspect, the length of the DNA can be optimized based on the desired mobility in a gel and the number of markers, e.g. fluorophores, necessary for detection, where a single molecule of DNA carrying one or more markers is easier to detect than the analyte itself. In addition, methods performed using multiple species of compositions, where the length of the polymer comprising the detection moiety determines the mobility of each species, can be used to simultaneously identify and quantify the analytes of complex solutions within e.g., the same lane of a gel or the same capillary.

In another embodiment compositions are used in combination with antibody pairs that specifically recognize an analyte. In one aspect, the presence or absence of the analyte is detected by the coincidence of two or more compositions, each of which binds an antibody. As an example, Jarvius, et al (2006), discloses a version of this assay in which DNA molecules covalently bound to antibodies serve as primers for sequence specific enzymatic amplification, and where the resulting amplified DNA is digitally detected in low-profile hydrodynamic flow. Compositions of the present invention can be used similarly, where the detection moiety of each detector molecule serves as a primer for enzymatic amplification. In another aspect, pairs of compositions can be used to directly detect target analyte molecules without amplification. When imaged electronically, such as with a camera or other photodetector, the presence of two or more detection moieties within a particular spatial or temporal window may be distinguished from the presence of a single detection moiety by signal amplitude. In another aspect, detection moieties can carry distinguishable markers, such as fluorophores with different emission spectra, where the colors are present or coincident only in the instance where the analyte is present in a sample.

Samples

In one embodiment, a sample comprises one or more analytes. A sample can be derived from any subject of interest, including mammalian subjects and, e.g., human subjects, e.g., patients. A sample can include blood and other liquid samples of biological origin, solid tissue samples such as a biopsy specimen or tissue cultures or cells derived therefrom, and the progeny thereof. A sample can be of cancerous origin, e.g., breast cancer. A sample can comprise a single cell or more than a single cell. Samples can also have been manipulated in any way after their procurement, such as by treatment with reagents, solubilization, or enrichment for certain components, such as proteins or polynucleotides. Sample also encompasses a clinical sample, and also includes cells in culture, cell supernatants, and cell lysates.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1

### Design and Use of LG-268-NRE for Analyte Detection and Quantitation in a Sample

A. Construction and Expression of LG-268-NRE

The sequences for construction of LG-268-NRE were identified from Genbank and the scientific literature. Exemplary sequences are included in Table 1.

LG Immunoglobulin binding protein is Genbank Accession Number: S50809. References for LG include: Kihlberg, B. M., Sjobring, U., Kastern, W. and Bjorck, L. “Protein LG: a hybrid molecule with unique immunoglobulin binding properties”, J. Biol. Chem. 267 (35), 25583-25588 (1992), and Ian Burbulis, Kumiko Yamaguchi, Richard Yu, Orna Resnekov & Roger Brent “Quantifying small numbers of antibodies with a ‘near-universal’ protein-DNA chimera”, Nature Methods—4, 1011-1013 (2007).

268 Zinc Finger is accession number (for the peptide sequence): 1AAY_A. References for 268 include: Pavletich, N. P. and Pabo, C. O. “Zinc finger-DNA recognition: crystal structure of a Zif268-DNA complex at 2.1 A”, Science 252 (5007), 809-817 (1991).

The NRE Zinc Finger amino acid sequence is published in: JIN-SOO KIM AND CARL O. PABO, “Getting a handhold on DNA: Design of poly-zinc finger proteins with femtomolar dissociation constants”, Proc. Natl. Acad. Sci. USA, Vol. 95, pp. 2812-2817, March 1998.

The LG-268-NRE sequence was then designed in silico, and a nucleic acid cassette of the sequence was chemically synthesized by Blue Heron Biotechnology (Blue Heron). The amino acid sequence was provided to Blue Heron for gene synthesis of LG-268-NRE and is shown in Table 1 (SEQ ID NO:6).

The LG-268-NRE cassette was cloned into pET20b(+) (Novagen), expressed in E. coli strain Rosetta gami (Novagen) using standard conditions, induced with 0.1 mM IPTG, and purified using the C terminal His tag on a Qiagen BioSprint nickel-NTA column per the manufacturer's instructions. LG-268-NRE (with His tag) was maintained in Tris-Glycine stock solution at 0.1 M.

B. Experimental Use of LG-268-NRE for Detection of an Analyte in a Sample

DNA detection moieties of different lengths were created. Each included the 268-NRE DNA binding site; the sequences are shown in Table 1 (SEQ ID NOS: 7-9). The DNA detection moieties include the 268-NRE DNA binding site sequence: GCGGGTGCGACTTGGGAA. All DNA detection moieties were chemically synthesized and assembled by annealing two single-stranded oligonucleotides. The 50-mer was purified by the vendor (Operon); the 80-mer and 100-mer were annealed using 1 cycle and were used without purification.

Stock solutions of LG-268-NRE (0.1 mM), the DNA detection moiety (500 ng/μl for a 50-mer and 1 μg/μl for an 80- or 100-mers), the target analytes (Bovine IgG antibodies (Equitech Bio, B60-0010; Final concentration of 1 mg/ml), and loading buffer (Jule Tris-Glycine running buffer; Jule Inc.; Catalog Number: TGNRB10X) were combined at the same time and mixed in a microfuge tube in a final reaction volume of 75 μl. Final concentrations were as follows: 0.03 mM for LG-268-NRE, 100 ng/75 μl for the 50-mer, and 10 μg/75 μl for the 80- or 100-mers. The combined mixture was then allowed to sit for 15-60 minutes at room temperature (approximately 20-22° C.). Appropriate volumes of the controls and the mixed solution were then loaded into wells of acrylamide (50 μl) or agarose gels (75 μl) and run as described below. The agarose gels and the staining buffer are made with Jule Tris-Glycine running buffer (Jule Inc.; Catalog Number: TGNRB10X). The binding reactions are also performed with a range of protein concentrations and/or in the presence of salt, e.g., 50-100 mM KCl or NaCl. Molecular weight standards were: Bioline Hyperladder 1: BIO-33053; Hyperladder 2: BIO-33054; and Hyperladder 3: BIO-33055.

FIG. 4 shows the results of a mobility shift assay run in a 4-20% gradient pre-cast acrylamide gel (Jule Inc.) for 75 minutes at a fixed current of 35 mA using a Tris Glycine running buffer (Jule Inc.; Catalog Number: TGNRB10X). FIG. 4 is an image of a gel stained with SYBR Gold (Invitrogen, S-11494) illuminated on a UV transilluminator, and photographed through a SYBR photographic filter (Invitrogen), which fluoresces in when bound to DNA, demonstrating the principal of mobility shift for analyte detection. Lane 1 is a size standard, Hyperladder II. Lane 2 contains a 50-mer DNA detection moiety plus LG-268-NRE. Lane 3 contains a 50-mer DNA detection moiety plus LG-268-NRE plus Bovine IgG antibodies (Equitech Bio, B60-0010; Final concentration of 1 mg/ml). Lane 4 contains an 80-mer DNA detection moiety. Lane 5 contains an 80-mer DNA detection moiety plus LG-268-NRE, where both the detection moiety and the detector complex are visible. Lane 6 contains an 80-mer DNA detection moiety plus LG-268-NRE plus Bovine IgG antibodies, where the unshifted detection moiety, the unbound detector complex, and the bound detector complex are visible as bands of varying width from the bottom to the top of the lane. Lane 7 contains a 100-mer DNA detection moiety. Lane 8 contains a 100-mer DNA detection moiety plus LG-268-NRE, where both the detection moiety and the detector complex are visible. Lane 9 contains a 100-mer DNA detection moiety plus LG-268-NRE plus Bovine IgG antibodies, where the unshifted detection moiety, the unbound detector complex, and the bound detector complex are visible as bands of varying width from the bottom to the top of the lane. ProDNA is LG-268-NRE and the bound DNA detection moiety.

The results demonstrate electrophoretic shifting of the detector molecule LG-268-NRE plus DNA detection moiety when bound to an analyte (Bovine IgG) (compare lanes 5 and 6; compare lanes 8 and 9).

A similar set of experiments were performed to demonstrate using a detector molecule (LG-268-NRE) plus 100-mer DNA detection moiety to detect streptavidin.

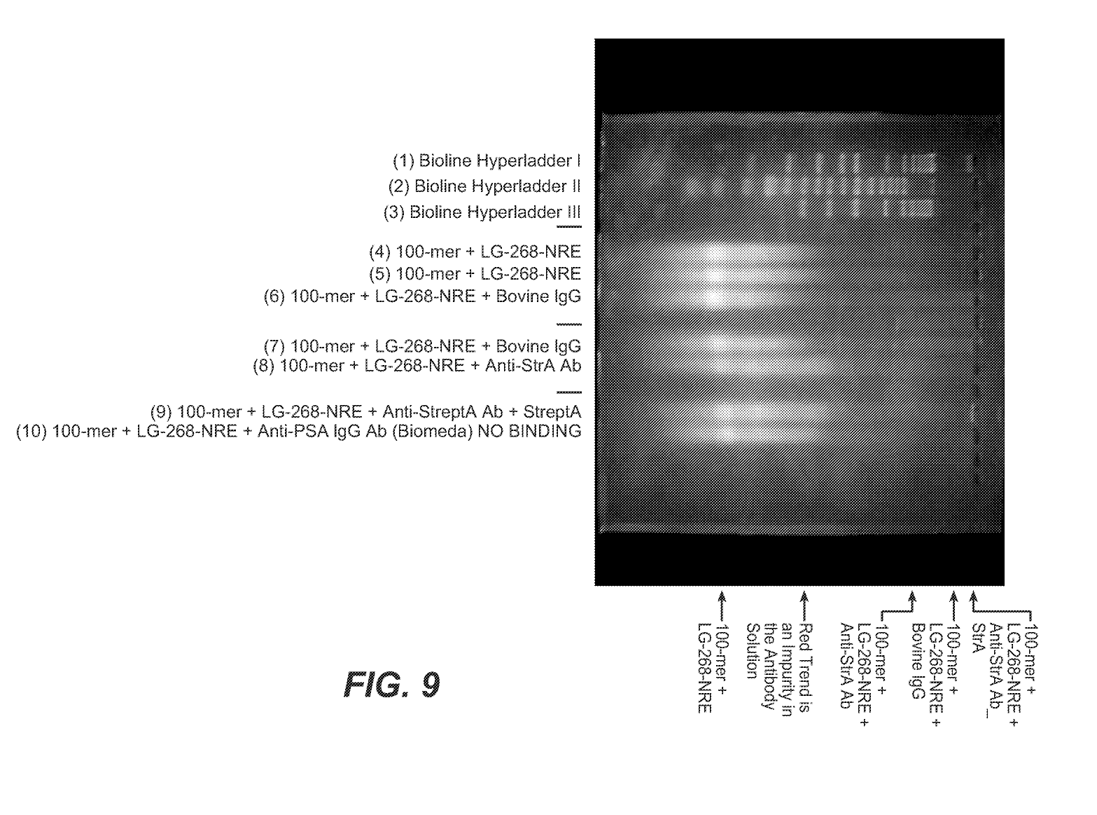

FIG. 9 shows a 4% agarose gel with a Tris Glycine running buffer (Jule Inc.) run for 180 min at a constant 100V. Lanes 1-3 are size standards, Bioline Hyperladder I, Bioline Hyperladder II, and Bioline Hyperladder III, respectively. Lane 4 contains a 100-mer DNA detection moiety plus LG-268-NRE. Lane 5 contains a 100-mer DNA detection moiety plus LG-268-NRE. Lane 6 contains a 100-mer DNA detection moiety plus LG-268-NRE plus Bovine IgG antibodies (see above). Lane 7 contains a 100-mer DNA detection moiety plus LG-268-NRE plus bovine IgG antibodies. Lane 8 contains a 100-mer DNA detection moiety plus LG-268-NRE plus anti-streptavidin A (StrA) antibodies (Anti StrA Ab: Genetex; GTX27241; final concentration is 300 μg/75 μL). Lane 9 contains a 100-mer DNA detection moiety plus LG-268-NRE plus anti-streptavidin A (StrA) antibodies plus Streptavidin A (SigmaAldrich; #85878; final concentration is 10 μg/75 μL). Lane 10 contains a 100-mer DNA detection moiety plus LG-268-NRE plus anti-PSA IgG control antibodies (Biomeda) that shows no binding. The gel was stained and imaged as above. The 100-mer DNA detection moiety here was prepared by annealing two 100-mer oligonucleotides without additional purification, which may account for the broad and blurry band.

### Example 2

### Design and Use of Streptavidin-Tetracycline Repressor (Strep-TetR) Protein for Analyte Detection and Quantitation in a Sample

A. Construction and Expression of Strep-TetR

The sequences for construction of Strep-TetR were identified from Genbank and the scientific literature.

Streptavidin is Genbank Accession Number: X03591.

Tetracycline repressor protein is GeneID: 5148505.

DNA sequence bound by TetR includes the sequence TCTATCATTGATAGG.

The Strep-TetR sequence is designed in silico, and a nucleic acid cassette of the sequence is chemically synthesized.

The Strep-TetR cassette is cloned into pET20b(+) (Novagen), expressed in E. coli strain Rosetta gami (Novagen), induced with 0.1 mM IPTG, and purified using the C terminal His tag on a Qiagen BioSprint nickel-NTA column per the manufacturer's instructions.

B. Experimental Use of Strep-TetR for Detection of an Analyte in a Sample

Strep-TetR and a DNA comprising detection moiety (50-mer, 80-mer, or a 100-mer) are maintained in Tris Glycine stock solutions. All DNA detection moieties are chemically synthesized and assembled by annealing two single-stranded oligonucleotides. The binding reactions are also performed with a range of protein concentrations and/or in the presence of salt, e.g., 50-100 mM KCl or NaCl. Typically, the DNA can be radiolabeled for detection, if needed.

Stock solutions of Strep-TetR, the DNA detection moiety, the target analyte(s), and loading buffer are combined and mixed in a microfuge tube. The combined mixture is then allowed to sit for 15-60 minutes at room temperature (approximately 20-22° C.). Appropriate volumes of the controls and the mixed solution are then loaded into wells of acrylamide (50 μl) or agarose gels (75 μl) and run at the appropriate voltage. Molecular weight standards are described above in Example 1. Samples run are as follows: lane 1: Molecular weight standard; lane 2: DNA detection moiety only; lane 3: DNA detection moiety plus Strep-TetR; lane 4: DNA detection moiety plus Strep-TetR plus sample with target analyte; lane 5: DNA detection moiety plus Strep-TetR plus sample with analyte that should not be bound by Strep.

By comparing lanes 3 and 4, and observing the band shift, one can detect the presence of the target analyte, e.g., biotin, in the sample.

### Example 3

### Design and Use of Streptavidin-Lac Repressor (Strep-Lac) Protein for Analyte Detection and Quantitation in a Sample

A. Construction and Expression of Strep-Lac

The sequences for construction of Strep-Lac were identified from Genbank and the scientific literature.

Streptavidin is Genbank Accession Number: X03591.

Lac repressor protein is GeneID: 4714237.

DNA sequence recognized by Lac includes a lac operon sequence and the sequences TATAAT and TTGACA.

The Strep-Lac sequence is designed in silico, and a nucleic acid cassette of the sequence is chemically synthesized.

The Strep-Lac cassette is cloned into pET20b(+) (Novagen), expressed in E. coli strain Rosetta gami (Novagen), induced with 0.1 mM IPTG, and purified using the C terminal His tag on a Qiagen BioSprint nickel-NTA column per the manufacturer's instructions.

B. Experimental Use of Strep-Lac for Detection of an Analyte in a Sample

Strep-Lac and a DNA comprising detection moiety (50-mer, 80-mer, or a 100-mer) are maintained in Tris Glycine stock solutions. All DNA detection moieties are chemically synthesized and assembled by annealing two single-stranded oligonucleotides. The binding reactions are also performed with a range of protein concentrations and/or in the presence of salt, e.g., 50-100 mM KCl or NaCl. Typically, the DNA can be radiolabeled for detection, if needed.

Stock solutions of Strep-Lac, the DNA detection moiety, the target analyte(s), and loading buffer are combined and mixed in a microfuge tube. The combined mixture is then allowed to sit for 15-60 minutes at room temperature (approximately 20-22° C.). Appropriate volumes of the controls and the mixed solution are then loaded into wells of acrylamide (50 μl) or agarose gels (75 μl) and run at the appropriate voltage. Molecular weight standards are described above in Example 1. Samples run are as follows: lane 1: Molecular weight standard; lane 2: DNA detection moiety only; lane 3: DNA detection moiety plus Strep-TetR; lane 4: DNA detection moiety plus Strep-TetR plus sample with target analyte; lane 5: DNA detection moiety plus Strep-TetR plus sample with analyte that should not be bound by Strep.

By comparing lanes 3 and 4, and observing the band shift, one can detect the presence of the target analyte, e.g., biotin, in the sample.

### Example 4

### Design and Use of Streptavidin-Lambda Repressor (Strep-Lam) Protein for Analyte Detection and Quantitation in a Sample

A. Construction and Expression of Strep-Lam

The sequences for construction of Strep-Lam were identified from Genbank and the scientific literature.

Streptavidin is Genbank Accession Number: X03591.

Lambda repressor protein is: X00166.

DNA sequence recognized by Lam includes the sequence TATCACCGC.

The Strep-Lam sequence is designed in silico, and a nucleic acid cassette of the sequence is chemically synthesized.

The Strep-Lam cassette is cloned into pET20b(+) (Novagen), expressed in E. coli strain Rosetta gami (Novagen), induced with 0.1 mM IPTG, and purified using the C terminal His tag on a Qiagen BioSprint nickel-NTA column per the manufacturer's instructions.

B. Experimental Use of Strep-Lam for Detection of an Analyte in a Sample

Strep-Lam and a DNA comprising detection moiety (50-mer, 80-mer, or a 100-mer) are maintained in Tris Glycine stock solutions. All DNA detection moieties are chemically synthesized and assembled by annealing two single-stranded oligonucleotides. The binding reactions are also performed with a range of protein concentrations and/or in the presence of salt, e.g., 50-100 mM KCl or NaCl. Typically, the DNA can be radiolabeled for detection, if needed.

Stock solutions of Strep-Lam, the DNA detection moiety, the target analyte(s), and loading buffer are combined and mixed in a microfuge tube. The combined mixture is then allowed to sit for 15-60 minutes at room temperature (approximately 20-22° C.). Appropriate volumes of the controls and the mixed solution are then loaded into wells of acrylamide (50 μl) or agarose gels (75 μl) and run at the appropriate voltage. Molecular weight standards are described above in Example 1. Samples run are as follows: lane 1: Molecular weight standard; lane 2: DNA detection moiety only; lane 3: DNA detection moiety plus Strep-TetR; lane 4: DNA detection moiety plus Strep-TetR plus sample with target analyte; lane 5: DNA detection moiety plus Strep-TetR plus sample with analyte that should not be bound by Strep.

By comparing lanes 3 and 4, and observing the band shift, one can detect the presence of the target analyte, e.g., biotin, in the sample.

### Example 5

### Design and Use of Protective Antigen-Tetracycline Repressor (Prot-TetR) Protein for Analyte Detection and Quantitation in a Sample

A. Construction and Expression of Prot-TetR

The sequences for construction of Prot-TetR were identified from Genbank and the scientific literature.

Protective Antigen is Genbank Accession Number: 2820165.

Tetracycline repressor protein is GeneID: 5148505.

DNA sequence recognized by TetR includes the sequence TCTATCATTGATAGG.

The Prot-TetR sequence is designed in silico, and a nucleic acid cassette of the sequence is chemically synthesized.

The Prot-TetR cassette is cloned into pET20b(+) (Novagen), expressed in E. coli strain Rosetta gami (Novagen), induced with 0.1 mM IPTG, and purified using the C terminal His tag on a Qiagen BioSprint nickel-NTA column per the manufacturer's instructions.

B. Experimental Use of Prot-TetR for Detection of an Analyte in a Sample

Prot-TetR and a DNA comprising detection moiety (50-mer, 80-mer, or a 100-mer) are maintained in Tris Glycine stock solutions. All DNA detection moieties are chemically synthesized and assembled by annealing two single-stranded oligonucleotides. The binding reactions are also performed with a range of protein concentrations and/or in the presence of salt, e.g., 50-100 mM KCl or NaCl. Typically, the DNA can be radiolabeled for detection, if needed.

Stock solutions of Prot-TetR, the DNA detection moiety, the target analyte(s), and loading buffer are combined and mixed in a microfuge tube. The combined mixture is then allowed to sit for 15-60 minutes at room temperature (approximately 20-22° C.). Appropriate volumes of the controls and the mixed solution are then loaded into wells of acrylamide (50 μl) or agarose gels (75 μl) and run at the appropriate voltage. Molecular weight standards are described above in Example 1.

### Example 6

### Design and Use of Protective Antigen-Lac Repressor (Prot-Lac) Protein for Analyte Detection and Quantitation in a Sample

A. Construction and Expression of Prot-Lac

The sequences for construction of Prot-Lac were identified from Genbank and the scientific literature.

Protective Antigen is Genbank Accession Number: 2820165.

Lac repressor protein is GeneID: 4714237.

DNA sequence recognized by Lac includes a lac operon sequence and the sequences TATAAT and TTGACA.

The Prot-Lac sequence is designed in silico, and a nucleic acid cassette of the sequence is chemically synthesized.

The Prot-Lac cassette is cloned into pET20b(+) (Novagen), expressed in E. coli strain Rosetta gami (Novagen), induced with 0.1 mM IPTG, and purified using the C terminal His tag on a Qiagen BioSprint nickel-NTA column per the manufacturer's instructions.

B. Experimental Use of Prot-Lac for Detection of an Analyte in a Sample

Prot-Lac and a DNA comprising detection moiety (50-mer, 80-mer, or a 100-mer) are maintained in Tris Glycine stock solutions. All DNA detection moieties are chemically synthesized and assembled by annealing two single-stranded oligonucleotides. The binding reactions are also performed with a range of protein concentrations and/or in the presence of salt, e.g., 50-100 mM KCl or NaCl. Typically, the DNA can be radiolabeled for detection, if needed.

Stock solutions of Prot-Lac, the DNA detection moiety, the target analyte(s), and loading buffer are combined and mixed in a microfuge tube. The combined mixture is then allowed to sit for 15-60 minutes at room temperature (approximately 20-22° C.). Appropriate volumes of the controls and the mixed solution are then loaded into wells of acrylamide (50 μl) or agarose gels (75 μl) and run at the appropriate voltage. Molecular weight standards are described above in Example 1. Samples run are as follows: lane 1: Molecular weight standard; lane 2: DNA detection moiety only; lane 3: DNA detection moiety plus Prot-Lac; lane 4: DNA detection moiety plus Prot-Lac plus sample with target analyte; lane 5: DNA detection moiety plus Prot-Lac plus sample with analyte that should not be bound by Prot.

By comparing lanes 3 and 4, and observing the band shift, one can detect the presence of the target analyte, e.g., B. anthracis, in the sample.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES CITED

1. Ian Burbulis, et al., “Using protein-DNA chimeras to detect and count small numbers of molecules”, Nature Methods, 2005. 2(1): p. 31-37.
2. Robert Carlson and Ian Burbulis, “Chimeric Fusion Molecule for Sensitive Analyte Detection and Quantitation”, 2002, US Patent Application 20030198973.
3. Ian Burbulis, et al, “Quantifying small numbers of antibodies with a ‘near-universal’ protein-DNA chimera”, Nature Methods, 2007, 4(12)
4. Jonas Jarvius, et al, Digital quantification using amplified single-molecule detection, Nature Methods, 2006, 3(9).

TABLE I
SEQUENCES

| SEQ ID NO | DESCRIPTION | SEQUENCE |
|---|---|---|
| SEQ ID | Amino Acid | mkktaiaiav alagfatvaq aavenkeetp etpetdseee |
| NO: 1 | Sequence of | vtikanlifa ngstqtaefk gtfekatsea yayadtlkkd |
| | LG | ngeytvdvad kgytlnikfa gkektpeepk eevtikanli |
| | | yadgktqtae fkgtfeeata eayryadalk kdngeytvdv |
| | | adkgytlnik fagkektpee pkeevtikan liyadgktqt |
| | | aefkgtfeea taeayryadl lakengkytv dvadkgytln |
| | | ikfagkektp eepkeevtik anliyadgkt qtaefkgtfa |
| | | eataeayrya dllakengkyt adledggyt inirfagkkv |
| | | dekpeepmdt yklilngktl kgettteavd aataekvfkq |
| | | yandngvdge wtyddatktf tvtekpevid aseltpavtt |
| | | yklvingktl kgetttkavd aetaekafkq yandngvdgv |
| | | wtyddatktf tvtem |
| SEQ ID | Gene | atgaaaaaaaccgcgattgcgattgcggtggcgctggcgggctttgcgacc |
| NO: 2 | Sequence of | gtggcgcaggcggcggtggaaaacaaagaagaaaccccggaaaccccggaa |
| | LG | accgatagcgaagaagaagtgaccattaaagcgaacctgatttttgcgaac |
| | | ggcagcacccagaccgcggaatttaaaggcacctttgaaaaagcgaccagc |
| | | gaagcgtatgcgtatgcggataccctgaaaaaagataacggcgaatatacc |
| | | gtggatgtggcggataaaggctataccctgaacattaaatttgcgggcaaa |
| | | gaaaaaaccccggaagaaccgaaagaagaagtgaccattaaagcgaacctg |
| | | atttatgcggatggcaaaacccagaccgcggaatttaaaggcacctttgaa |
| | | gaagcgaccgcggaagcgtatcgctatgcggatgcgctgaaaaaagataac |
| | | ggcgaatataccgtggatgtggcggataaaggctataccctgaacattaaa |
| | | tttgcgggcaaagaaaaaaccccggaagaaccgaaagaagaagtgaccatt |
| | | aaagcgaacctgatttatgcggatggcaaaacccagaccgcggaatttaaa |
| | | ggcacctttgaagaagcgaccgcggaagcgtatcgctatgcggatctgctg |
| | | gcgaaagaaaacggcaaatataccgtggatgtggcggataaaggctatacc |
| | | ctgaacattaaatttgcgggcaaagaaaaaaccccggaagaaccgaaagaa |
| | | gaagtgaccattaaagcgaacctgatttatgcggatggcaaaacccagacc |
| | | gcggaatttaaaggcacctttgcggaagcgaccgcggaagcgtatcgctat |
| | | gcggatctgctggcgaaagaaaacggcaaatataccgcggatctggaagat |
| | | ggcggctataccattaacattcgctttgcgggcaaaaaagtggatgaaaaa |
| | | ccggaagaaccgatggatacctataaactgattctgaacggcaaaaccctg |
| | | aaaggcgaaaccaccaccgaagcggtggatgcggcgaccgcggaaaaagtg |
| | | tttaaacagtatgcgaacgataacggcgtggatggcgaatggacctatgat |
| | | gatgcgaccaaaacctttaccgtgaccgaaaaaccggaagtgattgatgcg |
| | | agcgaactgaccccggcggtgaccacctataaactggtgattaacggcaaa |
| | | accctgaaaggcgaaaccaccaccaaagcggtggatgcggaaaccgcggaa |
| | | aaagcgtttaaacagtatgcgaacgataacggcgtggatggcgtgtggacc |
| | | tatgatgatgcgaccaaaacctttaccgtgaccgaaatg |
| SEQ ID | Amino Acid | merpyacpve scdrrfsrsd eltrhiriht gqkpfqcric |
| NO: 3 | Sequence of | mrnfsrsdhl tthirthtgekpfacdigrk farsderkrh tkihl |
| | 268 | |
| SEQ ID | Amino Acid | merpyacpve scdrrfsrsd eltrhiriht gqkpfqcric |
| NO:4 | Sequence of | mrnfsrsdhl tthirthtgekpfacdigrk farsderkrh |
| | NRE | tkihlrqkdg ggserpyacp vescdrrfsq shdltkhiri |
| | | htgqkpfqcr icmrnfsdss klsrhirtht gekpfacdic |
| | | grkfarldnr tahtkihlrq kd |
| SEQ ID | Full Amino | mkktaiaiav alagfatvaq aavenkeetp etpetdseee |
| NO: 5 | Acid | vtikanlifa ngstqtaefk gtfekatsea yayadtlkkd |
| | Sequence of | ngeytvdvad kgytlnikfa gkektpeepk eevtikanli |
| | LG-268-NRE | yadgktqtae fkgtfeeata eayryadalk kdngeytvdv |
| | | adkgytlnik fagkektpee pkeevtikan liyadgktqt |
| | | aefkgtfeea taeayryadl lakengkytv dvadkgytln |
| | | ikfagkektp eepkeevtik anliyadgkt qtaefkgtfa |
| | | eataeayrya dllakengky tadledggyt inirfagkkv |
| | | dekpeepmdt yklilngktl kgettteavd aataekvfkq |
| | | yandngvdge wtyddatktf tvtekpevid aseltpavtt |
| | | yklvingktl kgetttkavd aetaekafkq yandngvdgv |
| | | wtyddatktf tvtem |
| | | merpyacpve scdrrfsrsd eltrhiriht gqkpfqcric |
| | | mrnfsrsdhl tthirthtge kpfacdigrk farsderkrh |
| | | tkihlrqkdg ggserpyacp vescdrrfsq shdltkhiri |
| | | htgqkpfqcr icmrnfsdss klsrhirtht gekpfacdic |
| | | grkfarldnr tahtkihlrq kd |
| SEQ ID | Full Gene | ATGAAAAAAACTGCCATTGCCATCGCTGTCGCTCTCGCAGGATTCGCCACC |
| NO: 6 | Sequence of | GTCGCCCAAGCCGCTGTAGAAAACAAAGAAGAAACCCCAGAAACTCCGGAA |
| | LG-268-NRE | ACCGATTCAGAAGAGGAAGTGACTATTAAAGCCAATCTTATTTTTGCTAAC |
| | verified by | GGTTCAACACAGACAGCAGAATTTAAAGGTACTTTCGAAAAAGCCACATCA |
| | Blue Heron | GAAGCCTATGCGTATGCCGATACCCTGAAAAAAGACAATGGTGAGTATACC |
| | (plus 12 | GTCGACGTGGCAGACAAAGGCTATACATTAAACATTAAATTTGCAGGTAAA |
| | nucleotide | GAAAAAACGCCGGAAGAACCAAAAGAAGAAGTGACTATTAAAGCTAACCTT |
| | 5′ and 3′ | ATCTACGCCGATGGCAAGACCCAAACCGCGGAATTTAAAGGCACCTTCGAG |
| | fragments of | GAAGCTACAGCCGAAGCTTACCGCTACGCAGACGCCCTGAAAAAAGATAAT |
| | the cloning | GGCGAATATACCGTTGACGTAGCAGACAAAGGTTATACCCTGAACATTAAA |
| | vector, | TTTGCAGGTAAAGAGAAAACCCCCGAAGAACCGAAAGAAGAAGTTACGATT |
| | pUCminusMCS), | AAAGCCAACCTGATTTATGCAGATGGAAAAACCCAGACAGCTGAATTCAAA |
| | with the | GGGACGTTCGAAGAAGCAACAGCAGAAGCATATCGTTACGCAGATTTATTA |
| | codon usage | GCTAAGGAAAACGGTAAATATACGGTAGATGTTGCCGATAAAGGGTACACT |
| | | TTGAATATTAAATTCGCAGGCAAAGAAAAAACTCCAGAAGAACCAAAAGAA |
| | | GAAGTAACGATTAAAGCTAACCTCATCTATGCAGATGGTAAAACCCAGACA |
| | | GCAGAATTTAAAGGCACATTCGCAGAAGCCACCGCAGAAGCTTATCGTTAT |
| | | GCTGATTTGCTGGCTAAAGAAAATGGTAAATATACGGCAGACTTAGAAGAT |
| | | GGGGGTTACACCATTAACATTCGTTTCGCGGGAAAAAAAGTGGACGAAAAA |
| | | CCTGAGGAACCTATGGATACGTATAAATTAATCCTGAATGGTAAAACTTTG |
| | | AAAGGCGAGACCACAACAGAAGCAGTCGATGCAGCAACCGCTGAAAAAGTC |
| | | TTTAAACAATATGCTAATGATAATGGCGTAGACGGAGAATGGACCTATGAT |
| | | GATGCTACTAAAACCTTTACCGTTACTGAAAAACCTGAAGTTATCGATGCG |
| | | TCGGAACTGACTCCGGCTGTAACTACGTATAAACTCGTCATTAACGGCAAA |
| | | ACATTAAAAGGAGAAACCACCACCAAAGCAGTAGACGCCGAAACGGCCGAG |
| | | AAAGCCTTCAAACAGTACGCAAACGATAACGGTGTCGACGGCGTCTGGACC |
| | | TATGACGATGCTACGAAAACCTTTACCGTAACCGAAATGATGGAACGCCCG |
| | | TACGCCTGTCCCGTAGAAAGCTGTGACCGCCGTTTCTCTCGCTCCGATGAA |
| | | CTCACCCGCCACATCCGTATTCACACCGGTCAGAAACCGTTTCAATGTCGC |
| | | ATCTGTATGCGCAACTTTTCTCGTTCAGATCACTTAACCACCCATATCCGC |
| | | ACTCATACCGGGGAAAAACCTTTTGCGTGTGACATCGGACGTAAATTCGCC |
| | | CGCTCTGATGAACGTAAACGTCACACGAAAATTCATCTGCGCCAAAAAGAC |
| | | GGGGGCGGTAGCGAACGCCCGTATGCATGCCCAGTAGAATCATGTGATCGC |
| | | CGCTTTTCTCAATCCCATGACCTCACGAAACACATTCGCATTCATACCGGA |
| | | CAAAAACCGTTTCAATGTCGTATTTGCATGCGTAATTTCAGTGACTCTTCT |
| | | AAATTGTCCCGTCATATTCGTACACATACTGGTGAGAAACCTTTTGCCTGT |
| | | GATATCTGTGGTCGGAAATTCGCGCGCTTAGACAACCGCACGGCCCACACG |
| | | AAAATTCATTTGCGTCAGAAAGACCTCGAGATCGCT |
| SEQ ID | DNA | TAGTGAGTCGTATTAATTTCAGAATTCATTTCAAGGGTTCAGCGTGGGCG |
| NO: 7 | detection | |
| | moiety 50-mer | |
| SEQ ID | DNA | TTTTGGGGTTGATAGAAAAGATAGAGGGTATAGTGAGTCGTATTAATTTCA |
| NO: 8 | detection | GAATTCATTTCAAGGGTTCAGCGTGGGCG |
| | moiety 80-mer | |
| SEQ ID | DNA | CCCACTTCATTGTGCCAAAGTTTTGGGGTTGATAGAAAAGATAGAGGGTAT |
| NO: 9 | detection | AGTGAGTCGTATTAATTTCAGAATTCATTTCAAGGGTTCAGCGTGGGCG |
| | moiety 100-mer | |

1. A composition for detection of a target analyte, comprising:
a binding domain expressed as a fusion protein with a linking domain, wherein said binding domain is capable of binding to the target analyte with an affinity of at least at least 10⁻⁷, at least 10⁻⁸, or at least 10⁻⁹M; and
wherein said linking domain comprises a nucleic acid binding protein capable of noncovalently binding to a detection moiety comprising a nucleic acid.

2. The composition of claim 1, wherein said binding domain comprises LG.

3. (canceled)

4. The composition of claim 1, wherein said nucleic acid binding protein comprises a zinc finger protein.

5. The composition of claim 1, wherein said linking domain comprises a plurality of nucleic acid binding proteins.

6. The composition of claim 5, wherein said plurality of nucleic acid binding proteins comprises 268 and NRE.

7. The composition of claim 1, wherein said target analyte is capable of specifically binding a second target analyte.

8. The composition of claim 7, wherein said target analyte is an IgG antibody.

9. The composition of claim 7, wherein said second target analyte is an antigen.

10. The composition of claim 1, further comprising the detection moiety bound to said linking domain by a noncovalent bond to form a detector.

11. The composition of claim 1, wherein said binding domain comprises an antibody or a single chain fragment variable (scFv) protein.

12. The composition of claim 1, wherein said target analyte comprises an antibody, an IgG antibody, or an Fc fragment of an antibody.

13. The composition of claim 10, wherein said detection moiety comprises DNA.

14. (canceled)

15. (canceled)

16. The composition of claim 13, wherein said DNA comprises a nucleic acid restriction enzyme recognition site.

17. The composition of claim 10, wherein said detection moiety comprises a nucleic acid binding protein site.

18. (canceled)

19. (canceled)

20. The composition of claim 10, wherein said detection moiety comprises a nucleic acid comprising the DNA binding site for 268-NRE.

21. The composition of claim 10, wherein said detection moiety further comprises a marker.

22. The composition of claim 21, wherein said marker comprises a gold particle, a metal particle, a silver particle, a dye, an isotope, a fluorophore, a magnetic particle, or a quantum dot.

23. The composition of claim 10, wherein said detection moiety further comprises a plurality of distinct markers.

24. A method for determining the presence or absence of an analyte in a sample, comprising:
binding the composition of claim 1 to a detection moiety to form a detector;
acquiring the sample;
combining the sample with the detector, wherein said combining results in binding of said analyte by said detector; and
determining the presence of said analyte in said sample.

25-52. (canceled)


SEQUENCE LISTING (11 sequences)

SEQ ID NO: 1 - protein, 455 aa, Finegoldia magna
MKKTAIAIAVALAGFATVAQAAVENKEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAGKKVDEKPEEPMDTYKLILNGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEM

SEQ ID NO: 2 - DNA, 1365 bp, Finegoldia magna
atgaaaaaaaccgcgattgcgattgcggtggcgctggcgggctttgcgaccgtggcgcaggcggcggtggaaaacaaagaagaaaccccggaaaccccggaaaccgatagcgaagaagaagtgaccattaaagcgaacctgatttttgcgaacggcagcacccagaccgcggaatttaaaggcacctttgaaaaagcgaccagcgaagcgtatgcgtatgcggataccctgaaaaaagataacggcgaatataccgtggatgtggcggataaaggctataccctgaacattaaatttgcgggcaaagaaaaaaccccggaagaaccgaaagaagaagtgaccattaaagcgaacctgatttatgcggatggcaaaacccagaccgcggaatttaaaggcacctttgaagaagcgaccgcggaagcgtatcgctatgcggatgcgctgaaaaaagataacggcgaatataccgtggatgtggcggataaaggctataccctgaacattaaatttgcgggcaaagaaaaaaccccggaagaaccgaaagaagaagtgaccattaaagcgaacctgatttatgcggatggcaaaacccagaccgcggaatttaaaggcacctttgaagaagcgaccgcggaagcgtatcgctatgcggatctgctggcgaaagaaaacggcaaatataccgtggatgtggcggataaaggctataccctgaacattaaatttgcgggcaaagaaaaaaccccggaagaaccgaaagaagaagtgaccattaaagcgaacctgatttatgcggatggcaaaacccagaccgcggaatttaaaggcacctttgcggaagcgaccgcggaagcgtatcgctatgcggatctgctggcgaaagaaaacggcaaatataccgcggatctggaagatggcggctataccattaacattcgctttgcgggcaaaaaagtggatgaaaaaccggaagaaccgatggatacctataaactgattctgaacggcaaaaccctgaaaggcgaaaccaccaccgaagcggtggatgcggcgaccgcggaaaaagtgtttaaacagtatgcgaacgataacggcgtggatggcgaatggacctatgatgatgcgaccaaaacctttaccgtgaccgaaaaaccggaagtgattgatgcgagcgaactgaccccggcggtgaccacctataaactggtgattaacggcaaaaccctgaaaggcgaaaccaccaccaaagcggtggatgcggaaaccgcggaaaaagcgtttaaacagtatgcgaacgataacggcgtggatggcgtgtggacctatgatgatgcgaccaaaacctttaccgtgaccgaaatg

SEQ ID NO: 3 - protein, 85 aa, Mus musculus
MERPYACPVESCDRRFSRSDELTRHIRIHTGQKPFQCRICMRNFSRSDHLTTHIRTHTGEKPFACDIGRKFARSDERKRHTKIHL

SEQ ID NO: 4 - protein, 182 aa, Artificial Sequence
  Description of Artificial Sequence Synthetic polypeptide
MERPYACPVESCDRRFSRSDELTRHIRIHTGQKPFQCRICMRNFSRSDHLTTHIRTHTGEKPFACDIGRKFARSDERKRHTKIHLRQKDGGGSERPYACPVESCDRRFSQSHDLTKHIRIHTGQKPFQCRICMRNFSDSSKLSRHIRTHTGEKPFACDICGRKFARLDNRTAHTKIHLRQKD

SEQ ID NO: 5 - protein, 637 aa, Artificial Sequence
  Description of Artificial Sequence Synthetic polypeptide
MKKTAIAIAVALAGFATVAQAAVENKEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAGKKVDEKPEEPMDTYKLILNGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMMERPYACPVESCDRRFSRSDELTRHIRIHTGQKPFQCRICMRNFSRSDHLTTHIRTHTGEKPFACDIGRKFARSDERKRHTKIHLRQKDGGGSERPYACPVESCDRRFSQSHDLTKHIRIHTGQKPFQCRICMRNFSDSSKLSRHIRTHTGEKPFACDICGRKFARLDNRTAHTKIHLRQKD

SEQ ID NO: 6 - DNA, 1923 bp, Artificial Sequence
  Description of Artificial Sequence Synthetic polynucleotide
atgaaaaaaactgccattgccatcgctgtcgctctcgcaggattcgccaccgtcgcccaagccgctgtagaaaacaaagaagaaaccccagaaactccggaaaccgattcagaagaggaagtgactattaaagccaatcttatttttgctaacggttcaacacagacagcagaatttaaaggtactttcgaaaaagccacatcagaagcctatgcgtatgccgataccctgaaaaaagacaatggtgagtataccgtcgacgtggcagacaaaggctatacattaaacattaaatttgcaggtaaagaaaaaacgccggaagaaccaaaagaagaagtgactattaaagctaaccttatctacgccgatggcaagacccaaaccgcggaatttaaaggcaccttcgaggaagctacagccgaagcttaccgctacgcagacgccctgaaaaaagataatggcgaatataccgttgacgtagcagacaaaggttataccctgaacattaaatttgcaggtaaagagaaaacccccgaagaaccgaaagaagaagttacgattaaagccaacctgatttatgcagatggaaaaacccagacagctgaattcaaagggacgttcgaagaagcaacagcagaagcatatcgttacgcagatttattagctaaggaaaacggtaaatatacggtagatgttgccgataaagggtacactttgaatattaaattcgcaggcaaagaaaaaactccagaagaaccaaaagaagaagtaacgattaaagctaacctcatctatgcagatggtaaaacccagacagcagaatttaaaggcacattcgcagaagccaccgcagaagcttatcgttatgctgatttgctggctaaagaaaatggtaaatatacggcagacttagaagatgggggttacaccattaacattcgtttcgcgggaaaaaaagtggacgaaaaacctgaggaacctatggatacgtataaattaatcctgaatggtaaaactttgaaaggcgagaccacaacagaagcagtcgatgcagcaaccgctgaaaaagtctttaaacaatatgctaatgataatggcgtagacggagaatggacctatgatgatgctactaaaacctttaccgttactgaaaaacctgaagttatcgatgcgtcggaactgactccggctgtaactacgtataaactcgtcattaacggcaaaacattaaaaggagaaaccaccaccaaagcagtagacgccgaaacggccgagaaagccttcaaacagtacgcaaacgataacggtgtcgacggcgtctggacctatgacgatgctacgaaaacctttaccgtaaccgaaatgatggaacgcccgtacgcctgtcccgtagaaagctgtgaccgccgtttctctcgctccgatgaactcacccgccacatccgtattcacaccggtcagaaaccgtttcaatgtcgcatctgtatgcgcaacttttctcgttcagatcacttaaccacccatatccgcactcataccggggaaaaaccttttgcgtgtgacatcggacgtaaattcgcccgctctgatgaacgtaaacgtcacacgaaaattcatctgcgccaaaaagacgggggcggtagcgaacgcccgtatgcatgcccagtagaatcatgtgatcgccgcttttctcaatcccatgacctcacgaaacacattcgcattcataccggacaaaaaccgtttcaatgtcgtatttgcatgcgtaatttcagtgactcttctaaattgtcccgtcatattcgtacacatactggtgagaaaccttttgcctgtgatatctgtggtcggaaattcgcgcgcttagacaaccgcacggcccacacgaaaattcatttgcgtcagaaagacctcgagatcgct

SEQ ID NO: 7 - DNA, 50 bp, Artificial Sequence
  Description of Artificial Sequence Synthetic oligonucleotide
tagtgagtcgtattaatttcagaattcatttcaagggttcagcgtgggcg

SEQ ID NO: 8 - DNA, 80 bp, Artificial Sequence
  Description of Artificial Sequence Synthetic oligonucleotide
ttttggggttgatagaaaagatagagggtatagtgagtcgtattaatttcagaattcatttcaagggttcagcgtgggcg

SEQ ID NO: 9 - DNA, 100 bp, Artificial Sequence
  Description of Artificial Sequence Synthetic polynucleotide
cccacttcattgtgccaaagttttggggttgatagaaaagatagagggtatagtgagtcgtattaatttcagaattcatttcaagggttcagcgtgggcg

SEQ ID NO: 10 - DNA, 18 bp, Artificial Sequence
  Description of Artificial Sequence Synthetic oligonucleotide
gcgggtgcgacttgggaa

SEQ ID NO: 11 - DNA, 15 bp, Artificial Sequence
  Description of Artificial Sequence Synthetic oligonucleotide
tctatcattgatagg